(19) United States
(12) Patent Application Publication
(10) Pub. No.: US 2023/0181070 A1
(43) Pub. Date: Jun. 15, 2023

(54) SINGLE UNIT BODILY FLUID COLLECTION, MIXING, AND TESTING UNIT

(71) Applicant: Seven.me, Inc., Oakland, CA (US)

(72) Inventors: Vikash Rugoobur, Oakland, CA (US); Polina Segalova, San Francisco, CA (US)

(73) Assignee: Seven.me, Inc., Oakland, CA (US)

(21) Appl. No.: 17/571,461

(22) Filed: Jan. 8, 2022

Related U.S. Application Data
(60) Provisional application No. 63/288,431, filed on Dec. 10, 2021.

(51) Int. Cl. A61B 5/15; A61B 5/157

(52) U.S. Cl. CPC A61B 5/150755; A61B 5/150236; A61B 5/157; A61B 5/150221

(57) ABSTRACT

A single unit bodily fluid collection and mixing device that is used for collecting a specified amount of bodily fluid, mixing the bodily fluid with a specified amount of solution, mixing the bodily fluid and solution in a contained volume to prevent contamination, and applying the resulting mixture to a lateral flow test surface. The unit is a single device, and does not require multiple pieces or systems to collect patient bodily fluid, measure out solution, combine the patient bodily fluid, and apply the mixture to a testing surface. Rather, the single unit already contains the desired amount of solution, a collection tube receives a specified amount of bodily fluid from a patient, and the single device can be used to mix the two fluids together.

### CROSS REFERENCE TO RELATED APPLICATIONS

This application claims the priority benefit of U.S. Provisional Application Ser. No. 63/288,431, titled “SINGLE UNIT BODILY FLUID COLLECTION, MIXING, AND TESTING UNIT,” filed Dec. 10, 2021, the disclosure of which are incorporated herein by reference.

### BACKGROUND

As medicine advances, there are more opportunities for patients to do self testing in the comfort of their own home. Doing tests at home is more convenient, can save a patient money, and potentially be performed and provide results faster than if performed at a medical provider.

There are several disadvantages, however, to doing self testing at home. First of all, several tests require a certain volume of blood of a user to be mixed with a predetermined amount of liquid and then tested. For a user to get the right amount of blood, and to combine the correct blood volume with the correct amount of predetermined liquid can be very difficult. Most self testing systems involve several parts and steps, including a first system to obtain blood, a second system to measure out the blood, a third system to measure out the solution, a fourth system to combine the fluids, and then a system for applying the mixed solutions to a test surface. Additionally, when the mixing occurs at a home environment, there is a greater risk for contaminating the resulting mixture. As a result, many, if not all, have several steps that are prone to error, and most if not all at home testing systems are not as accurate as medical in-house systems.

What is needed is an improved system for allowing patients to take medical tests remotely.

### SUMMARY

The present technology, roughly described, is a single unit bodily fluid collection and mixing device that is used for collecting a specified amount of bodily fluid, mixing the bodily fluid with a specified amount of solution, mixing the bodily fluid and solution in a contained volume to prevent contamination, and applying the resulting mixture to a lateral flow test surface. The unit is a single device, and does not require multiple pieces or systems to collect patient bodily fluid, measure out solution, combine the patient bodily fluid, and apply the mixture to a testing surface. Rather, the single unit already contains the desired amount of solution, a collection tube receives a specified amount of bodily fluid from a patient, and the single device can be used to mix the two fluids together.

In embodiments, a device for preparing a bodily fluid sample to be tested can include a piston, a sleeve, a collection tube, and a reservoir. The sleeve is configured to receive the piston. The piston can extend outward from a first end of the sleeve and is slideably coupled to the sleeve. A collection tube extends from a second end of the sleeve to a first end of the piston. The collection tube has an aperture at a first end of the collection tube for receiving a bodily fluid from a patient, and the bodily fluid is stored within the collection tube. A reservoir is coupled to the sleeve and includes a predetermined quantity of solution. The reservoir receives the collection tube when the piston is displaced into the sleeve. The collection tube is configured to release the bodily fluid into the reservoir when the piston is displaced into the sleeve.

### BRIEF DESCRIPTION OF FIGURES

FIG. 1 illustrates a single unit bodily fluid collection and mixing device.

FIG. 2 illustrates an exploded view of a single unit bodily fluid collection and mixing device.

FIG. 3 illustrates a single unit bodily fluid collection and mixing device with solution.

FIG. 4 illustrates a single unit bodily fluid collection and mixing device receiving a bodily fluid.

FIG. 5 illustrates a single unit bodily fluid collection and mixing device that has received bodily fluid.

FIG. 6 illustrates a single unit bodily fluid collection and mixing device with a piston being displaced into a sleeve.

FIG. 7 illustrates a single unit bodily fluid collection and mixing device with a piston completely displaced into a sleeve with bodily fluid from a collection tube mixed with solution in a reservoir.

FIG. 8 illustrates a single unit bodily fluid collection and mixing device having a cap removed from the device.

FIG. 9 illustrates a single unit bodily fluid collection and mixing device that is applying a mixture of bodily fluid and solution onto a lateral flow test sample unit.

FIG. 10 illustrates a method for receiving, mixing, and application of test solution for a lateral flow test.

### DETAILED DESCRIPTION

The present technology, in some embodiments, is a single unit bodily fluid collection and mixing device that is used for collecting a specified amount of bodily fluid, mixing the bodily fluid with a specified amount of solution, mixing the bodily fluid and solution in a contained volume to prevent contamination, and applying the resulting mixture to a lateral flow test surface. The unit is a single device, and does not require multiple pieces or systems to collect patient bodily fluid, measure out solution, combine the patient bodily fluid, and apply the mixture to a testing surface. Rather, the single unit already contains the desired amount of solution, a collection tube receives a specified amount of bodily fluid from a patient, and the single device can be used to mix the two fluids together.

In embodiments, the single unit includes pieces such as a piston, a sleeve, a valve, a collection tube, a reservoir, and a cap. The collection tube extends from an outer surface of a piston to a surface of the valve inside the device. A patient may apply bodily fluid to an aperture in the piston and into an inner volume of the collection tube. The inner volume of the collection tube is designed to have the exact desired volume for the bodily fluid required for the particular test. Once a patient fills the inner volume of the collection tube with bodily fluid, the piston can be displaced or moved into the sleeve. In some instances, the piston can be displaced when a patient depresses the outer surface of the piston towards the reservoir or bottom of the device.

As the piston is depressed into the sleeve, the piston moves the collection tube towards the reservoir, through the valve, such that the bodily fluid content of the collection tube is released into the reservoir. The bodily fluid in the reservoir is then mixed with the predetermined amount of solution in the reservoir, resulting in a mixture having the ideal amounts of bodily fluid and solution. Because the mixing is performed inside the device, there is a minimal chance of contaminating the solution by outside particles.

Once mixed, the mixture can be applied to a testing surface, for example as part of a lateral flow test. To apply the mixture to a testing surface, a cap on the outer surface of the reservoir can be removed, revealing an applicator to release the mixture onto the surface. In some instances, all or a portion of the single unit can be compressed to forcibly cause the mixture to exit the unit and onto a testing surface.

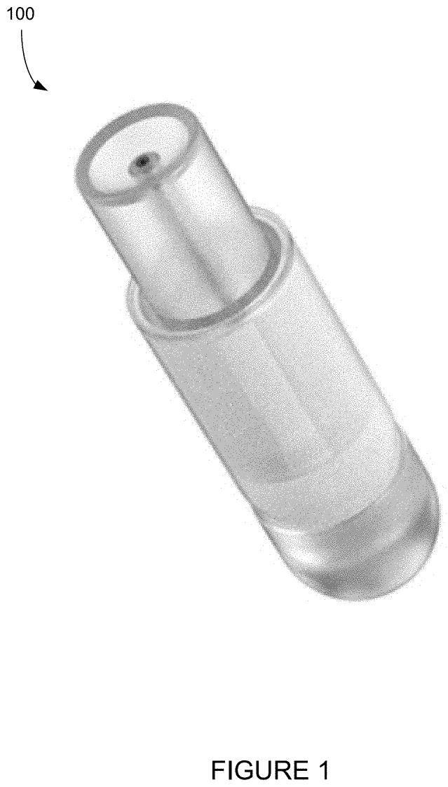

FIG. 1 illustrates a single unit bodily fluid collection and mixing device. The single unit device 100 of FIG. 1 is able to receive bodily fluid from a patient. In some instances, the bodily fluid may include blood, but may include other fluids such as plasma, saliva and other bodily fluids. The bodily fluid is received by the single device until a maximum volume is received. Once the maximum volume is received, a piston can be depressed through a sleeve, and the received bodily fluid is combined with a predetermined amount of solution. The received bodily fluid and predetermined solution is then mixed, for example by shaking the device. Once the mixer between the bodily fluid and solution is completely mixed, the solution may be dispersed by the device through an aperture or other mechanism. In some instances, a cap be removed from the bottom of the device which reveals an applicator for applying the mixed solution and bodily fluid onto a lateral flow test testing unit, such as a card, paper, or other lateral flow test surface.

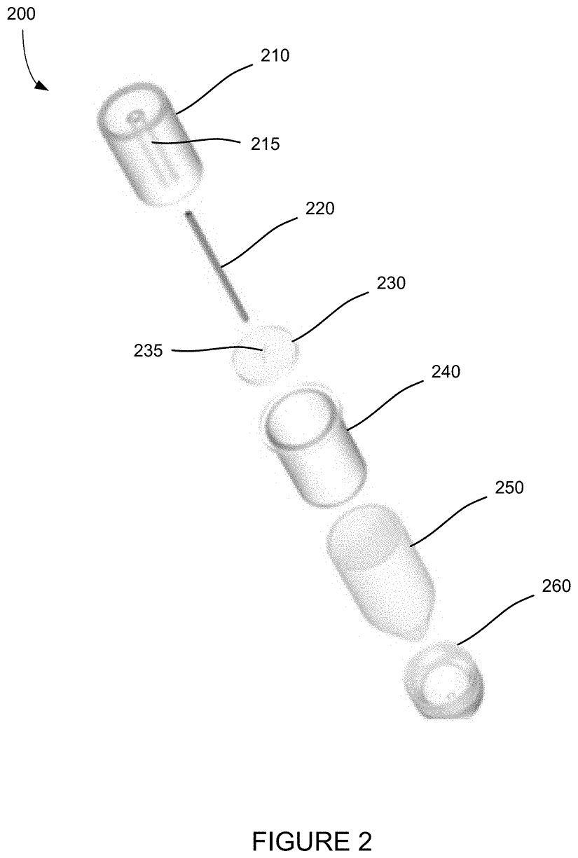

FIG. 2 illustrates an exploded view of a single unit bodily fluid collection and mixing device. The single unit 200 of FIG. 2 includes a piston 210, a cylindrical opening within the piston 215, a collection tube 220, a valve 230, a sleeve 240, a reservoir 250, and a cap 260.

The collection tube 220 extends through a cylindrical opening 215 within the piston from a surface of valve 230 to the top surface of piston 210. In some instances, the top of piston 210 may be concave or otherwise depressed, in which case the collection tube 220 would be flush with the appropriate portion of the surface of the piston. The other end of collection tube 220 can rest on valve 230, which is located inside sleeve 240. When piston 210 is displaced into sleeve 240, the collection tube 220 may extend through valve 230 at point 235 so that collection tube 220 protrudes through sleeve 240 and reservoir 250.

Once collection tube 220 is full of bodily fluid, whether it be blood, plasma, saliva, or some other fluid, piston 210 can be displaced through sleeve 240 by applying pressure to piston 210. The pressure may be applied by a machine, a user, or some other source of pressure. Once the piston is completely displaced into sleeve 240, up until valve 230, the bodily fluid within collection tube 220 is released into reservoir 250.

Reservoir 250 can include a predetermined amount of solution. The amount of solution contained in reservoir 250 can combine with the predetermined volume of bodily fluid within collection tube 220 to generate a perfect mixture for testing purposes. Once bodily fluid has exited collection tube 220 and enters reservoir 250, the liquids can be combined, for example by shaking the device. Once mixed, a cap 260 can be removed from the unit 200, which reveals an applicator tip at the bottom of the device. The mixture can then be provided through the applicator onto a testing surface. In some instances, the reservoir 250 may be squeezed, and is therefore flexible, to apply the mixture from the device to a surface.

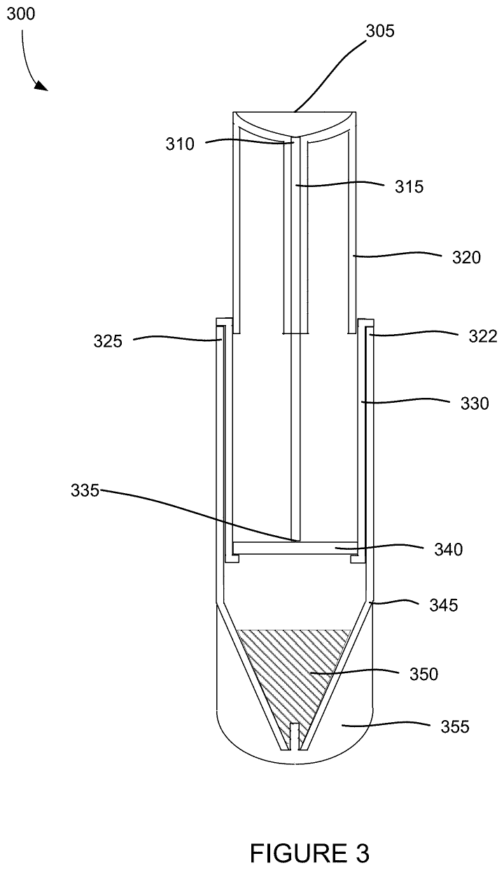

FIGS. 3-9 illustrate a single unit bodily fluid collection and mixing device in different stages of use. Each stage of use will be discussed with respect to FIGS. 3-9 below.

FIG. 3 illustrates a single unit bodily fluid collection and mixing device with solution. Similar to the device of FIG. 2, the single device of 300 includes a piston 320, a sleeve 330, a collection tube 315, a valve 340, a reservoir 345, solution 350 within reservoir 345, and cap 355. The single unit device 300 is designed to reduce the human error for bodily fluid collection, mixing, testing as part of a lateral flow test. The device is made as a single unit, such that a bodily fluid is received by, the bodily fluid is mixed within, and the solution and bodily fluid mixture is applied from the same singular device.

As will be discussed in more detail, a patient applies a bodily fluid through the first end of the collection tube 310, a piston 320 can then be depressed by user or other means to exert a force to displace collection tube 315 towards solution 350 within reservoir 345. When the collection tube 315 makes contact with the solution 350, the bodily fluid exits the collection tube and enters solution 350. The two fluids are then combined, and the user may shake or otherwise manipulate the singular device to mix the fluids further. Once mixed, a cap 355 or other tip mechanism can be removed from the device 300, and the mixed bodily fluid and's solution can be applied to a testing service.

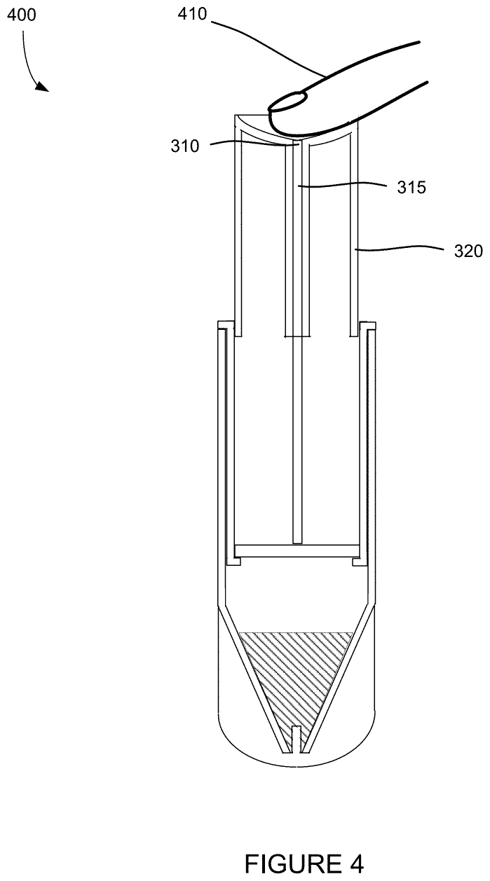

FIG. 4 illustrates a single unit bodily fluid collection and mixing device receiving a bodily fluid. The device 400 of FIG. 4 is illustrated with a patient's finger 410 depressed at a first end 310 of collection tube 315. A user may have a prick or other laceration on the patient's finger that allows blood to come from the user's finger into collection tube 315. The user may hold their finger at device 400 until blood has filled collection tube 315, from the bottom of the collection tube at the valve up to the end of the tube at the surface of the piston. The volume of the collection tube 315 is precisely designed to be the exact volume needed to combine with a solution in the reservoir of the device to perform the particular test.

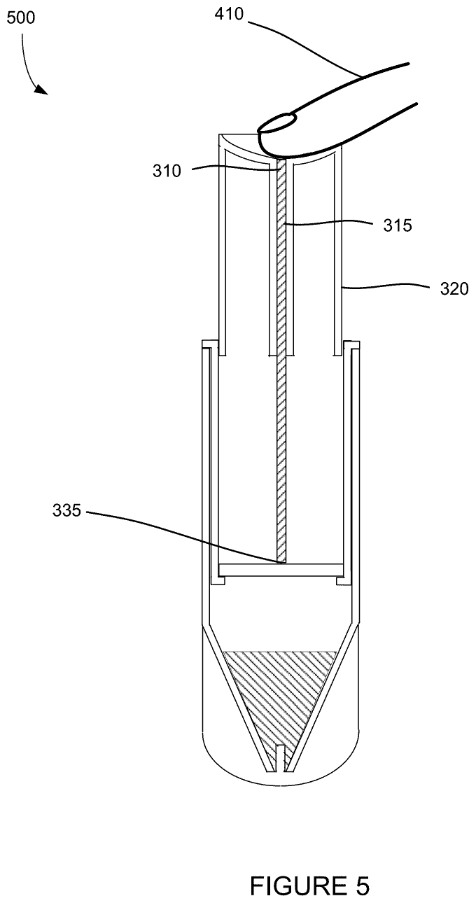

FIG. 5 illustrates a single unit bodily fluid collection and mixing device that has received bodily fluid. As shown in FIG. 5, collection tube 315 is full of bodily fluid from user, as bodily fluid has extended from a first end 310 to a second and 335 of the collection tube 315. In this case, the bodily fluid is blood received from the user's finger for 10. In some instances, a bodily fluid other than blood may be inserted in the collection tube, such as plasma, saliva, or some other bodily fluid from a patient. Regardless of the fluid type, the volume of the collection tube may be designed to receive the exact volume needed to combine with solution within a reservoir of the device.

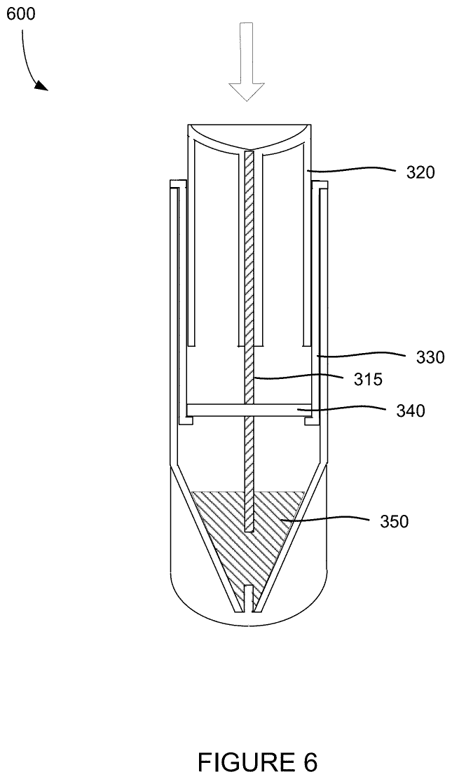

FIG. 6 illustrates a single unit bodily fluid collection and mixing device with a piston being displaced into a sleeve. Once collection tube 315 is full of bodily fluid, piston 320 may be depressed into sleeve 330. By depressing the piston into the sleeve, collection tube 315 penetrates valve 340 and extends towards solution 350. Once the piston 320 is oppressed all the way into sleeve 330, the bodily fluid from collection tube 315 reaches the reservoir and the bodily fluid exits the collection tube and enters the reservoir containing solution 350.

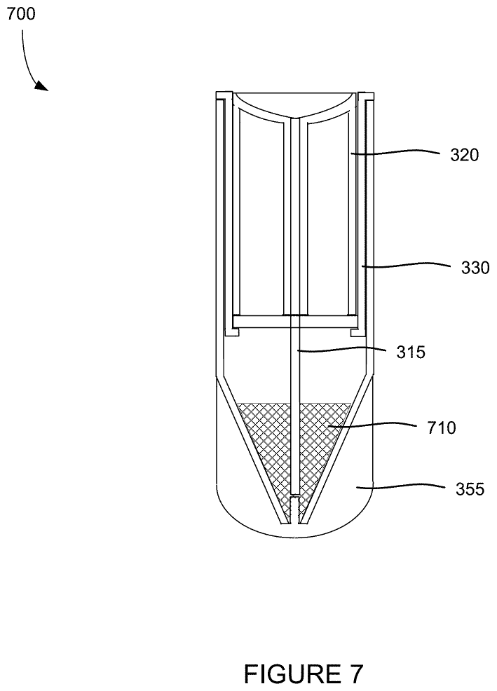

FIG. 7 illustrates a single unit bodily fluid collection and mixing device with a piston completely displaced into a sleeve with bodily fluid from a collection tube mixed with solution in a reservoir. As shown in FIG. 7, piston 320 is completely displaced into sleeve 330, such that the end of piston 320 is in contact with a surface of a filter 340. The collection tube 315 has penetrated the solution within the reservoir, the bodily fluid has left the collection tube and entered the solution, and the bodily fluid and solution are mixed to become mixture 710 of bodily fluid and solution. The mixture of bodily fluid and solution has the ideal amounts of bodily fluid and solution. The desired bodily fluid volume is obtained by having a user fill the collection tube, which has various precise directions for a particular test. The solution material is predetermined for the particular test and added by a manufacturer within the reservoir.

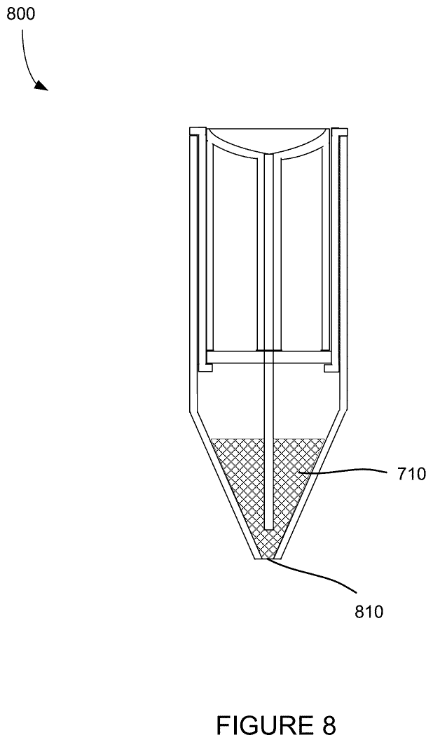

FIG. 8 illustrates a single unit bodily fluid collection and mixing device having a cap removed from the device. Once the solution and bodily fluid are mixed, a cap 355 is removed, resulting in the unit 800 of FIG. 8. Once the cap is removed, an opening 810 is revealed in the unit which allows the mixture of bodily fluid and solution to be applied to a testing surface as part of a lateral flow test.

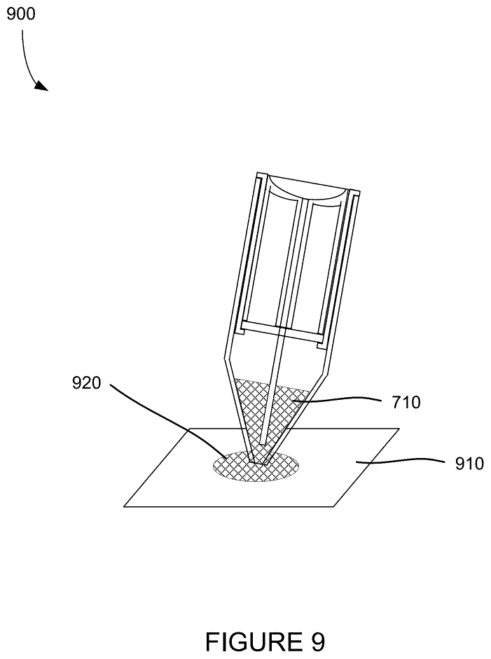

FIG. 9 illustrates a single unit bodily fluid collection and mixing device that is applying a mixture of bodily fluid and solution onto a lateral flow test sample unit. The singular device is positioned above a lateral flow test surface 910 such that the mixed solution 710 can be applied to the surface 910. As indicated, the mixture leaves the single unit and is deposited as test solution 920 on the surface 910.

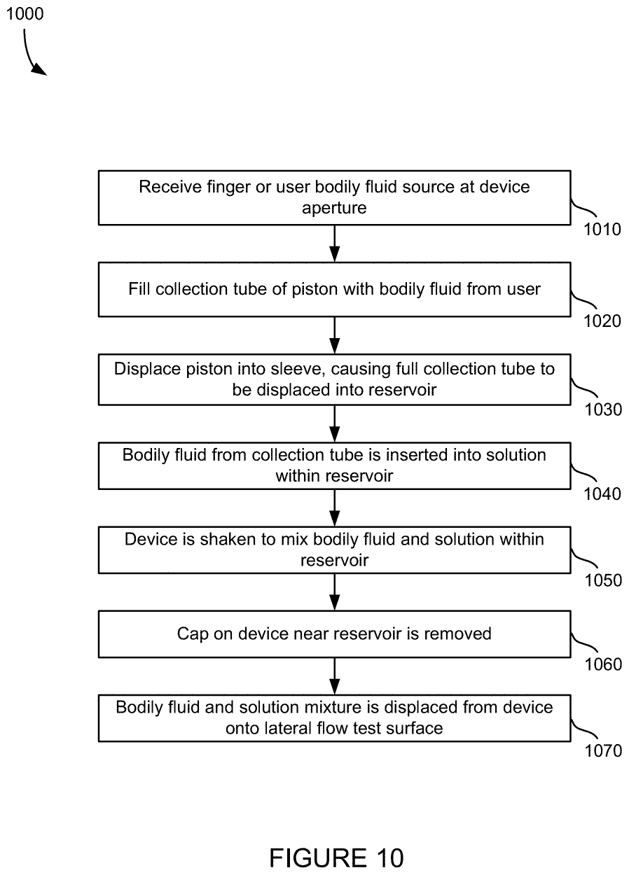

FIG. 10 illustrates a method for receiving, mixing, and application of test solution for a lateral flow test. The method of FIG. 10 begins with receiving a finger or other bodily fluid source at a device aperture at step 1010. The device aperture may be a first end of a collection tube at an end of the device. The collection tube is then filled with bodily fluid from a user at step 1020. The bodily fluid may include blood, plasma, saliva, or some other bodily fluid from a user.

Once the collection tube is full of bodily fluid, a piston is displaced into a sleeve at step 1030. By displacing the piston into the sleeve, the prescribed amount of bodily fluid with the collection tube is displaced into a reservoir at step 1030. The piston, for example, may be pushed down by a user such that the piston is displaced into the sleeve. As the piston is displaced into the sleeve, a collection tube may penetrate a valve such that the collection tube extends past the valve into a reservoir holding solution.

Bodily fluid from the collection tube is inserted into the solution within the reservoir at step 1040. As the collection tube is displaced towards the reservoir, bodily fluid within the collection tube is released into the solution within the reservoir. The device may then be shaken to mix the bodily fluid and the solution within the reservoir at step 1050.

Once mixed, a cap on the device the reservoir is removed at step 1060. The cap can be removed to reveal an aperture at the reservoir end of the device. Once the cap is removed, the reservoir may be squeezed in order to release the mixture of bodily fluid and solution. The bodily fluid and solution mixture is then displaced from the device onto a lateral flow test surface at step 1070. The test surface may be a lateral flow test surface aligned with the volumes of solution and bodily fluid stored within the collection tube.

Though the present device is described as a single unit for performing a single test, a single unit can be configured for multiple tests, in some embodiments. For example, the single unit may include multiple selectable paired pistons and reservoirs. A patient may select a paired piston and reservoir, provide the particular amount of bodily fluid, combine the provided bodily fluid with the prescribed amount of solution, mix the two, and provide the mixture to a test surface. In this embodiment, a user may select from one of multiple tests to perform for a single unit, and still experience the advantages of the present system, including but not limited to a single device for receiving bodily fluid, combining bodily fluid with solution, and doing so in a contained space to avoid contamination.

The foregoing detailed description of the technology herein has been presented for purposes of illustration and description. It is not intended to be exhaustive or to limit the technology to the precise form disclosed. Many modifications and variations are possible in light of the above teaching. The described embodiments were chosen to best explain the principles of the technology and its practical application to thereby enable others skilled in the art to best utilize the technology in various embodiments and with various modifications as are suited to the particular use contemplated. It is intended that the scope of the technology be defined by the claims appended hereto.

What is claimed is:

1. A device for preparing a bodily fluid sample to be tested, the device comprising:
a piston;
a sleeve configured to receive the piston, the piston extending outward from a first end of the sleeve and slideably coupled to the sleeve;
a collection tube extending from a second end of the sleeve to a first end of the piston, the collection tube having an aperture at a first end of the collection tube for receiving a bodily fluid from a patient, the bodily fluid stored within the collection tube; and
a reservoir coupled to the sleeve, the reservoir including a predetermined quantity of solution, the reservoir configured to receive the collection tube when the piston is displaced into the sleeve, the collection tube configured to release the bodily fluid into the reservoir when the piston is displaced into the sleeve.

2. The device of claim 1, wherein the piston, sleeve, collection tube and reservoir are part of a single device.

3. The device of claim 1, wherein the collection tube has an inner portion to receive the bodily fluid, the inner portion having a volume that collects a static volume of blood to be combined, within the single device, with the predetermined quantity of solution to be used for a particular test.

4. The device of claim 1, wherein the piston is configured to receive a bodily fluid from a finger of the patient through the aperture of the collection tube.

5. The device of claim 1, further comprising a valve, the valve displaced within the sleeve, the valve keeping the collection tube in place while the collection tube receives bodily fluid, the valve allowing the collection tube to pass through the valve when the piston is displaced into the sleeve.

6. The device of claim 5, the valve preventing any particles from outside the device to enter the reservoir except the bodily fluid from the collection tube.

7. The device of claim 1, wherein the piston is displaced into the sleeve when a pressure is exerted on the piston towards the sleeve.

8. The device of claim 1, further comprising a cap displaced over an outer surface of the device corresponding to the reservoir, the cap removable from the device, the reservoir including an aperture under the cap that allows a mixture of the solution and bodily fluid to be dispersed out of the device once the cap is removed.

9. The device of claim 8, wherein the mixture of bodily fluid and solution are combined in the reservoir and used to perform a lateral flow test.

10. The device of claim 1, wherein the inner surface of the sleeve and the outer surface of the piston have a similar shape.

11. The device of claim 10, wherein the inner surface of the sleeve and the outer surface of the piston have a circular shape.

12. The device of claim 1, wherein the bodily fluid and solution are mixed in the reservoir
